(19) United States
(12) Patent Application Publication
(10) Pub. No.: US 2003/0093046 A1
(43) Pub. Date: May 15, 2003

(54) Cover sheet for personal care products

(76) Inventors: HyungByum Kim, TaeJon-city (KR); EoYeon Hwang, Seoul (KR); HyungWoo Park, Kyung Ki-Do (KR); EunJung Kang, Seoul (KR)

Correspondence Address: KIMBERLY-CLARK WORLDWIDE, INC., 401 NORTH LAKE STREET, NEENAH, WI, 54956

(21) Appl. No.: 09/990,787

(22) Filed: Nov. 14, 2001

(51) Int. Cl. A61F 13/15

(52) U.S. Cl. 604/367

(57) ABSTRACT

There is provided a liner for personal care products having a hydrophilic first apertured nonwoven layer laminated with a hydrophobic second apertured nonwoven layer. The apertures may be aligned. The first layer may, further, be made of durably hydrophilic fibers and the second of non-durably hydrophilic fibers. The liner may be made by a spunlace process. The liner may further have a treatment applied to the hydrophilic layer, where the treatment is aloe, vitamin E, mineral oil, baking soda and combinations thereof. Also provided is a pantiliner having a liquid permeable liner, a liquid impervious baffle, and an absorbent core positioned therebetween. The liner has a hydrophilic first apertured nonwoven layer laminated according to a spunlace process with a hydrophobic second apertured nonwoven layer. The apertures of the first layer and the second layer may be aligned.

### BACKGROUND OF THE INVENTION

The present invention concerns liners, top sheets or cover sheets for personal care products like feminine hygiene products, diapers, training pants and the like.

Liners are designed to be permeable to liquid and to be non-irritating to the skin since they are the outermost layer of a personal care product and so in contact with the wearer. Liners feel soft to the skin and allow urine and menses to penetrate quite easily. Liners have been made from various materials including nonwoven webs, apertured films, foams and combinations thereof. The nonwovens and films may be made from synthetic polymers, including polyolefins like polyethylene and polypropylene. The nonwovens may also be made from natural fibers or combinations of natural and synthetic fibers. Liners may also be made from creped materials such as creped nonwoven webs.

Liners have advanced significantly over the years, though rewet of the wearer's skin and leakage, especially in the case of feminine hygiene products, remains an important issue. There remains a need, therefore, for a liner that will rapidly take in fluids like urine and menses and retard or prevent it from moving upwardly towards the wearer again.

### SUMMARY OF THE INVENTION

In response to the discussed difficulties and problems encountered in the prior art, a new liner has been developed wherein the liner has a hydrophilic first apertured nonwoven layer laminated with a hydrophobic second apertured nonwoven layer. The apertures may be aligned. The first layer may, further, be made of durably hydrophilic fibers and the second of non-durably hydrophilic fibers (later made hydrophobic). The liner may be made by a spunlace process. The liner may further have a treatment applied to the hydrophilic layer, where the treatment is aloe, vitamin E, mineral oil, baking soda and combinations thereof.

Another embodiment of the liner of the present invention has a first nonwoven layer made from staple, naturally hydrophilic fibers hydroentangled to form a laminate with a second nonwoven layer made from hydrophobic fibers, where the laminate is apertured with an area of 10 to 50 percent. The liner may further have a first layer made from hydrophilic fibers of rayon, pulp, cotton, naturally hydrophilic fibers, and mixtures thereof. The hydrophobic fibers may be made from polymers like polyolefins, polyesters, acrylics and mixtures thereof.

Also provided is a pantiliner having a liquid permeable liner, a liquid impervious baffle, and an absorbent core positioned therebetween. The liner has a hydrophilic first apertured nonwoven layer laminated according to a spunlace process with a hydrophobic second apertured nonwoven layer. The apertures of the first layer and the second layer may be aligned.

The liner of this invention may be used in other personal care products like diapers, training pants, disposable swim wear, absorbent underpants, adult incontinence products, bandages, veterinary and mortuary products, and feminine hygiene products like sanitary napkins.

Also disclosed is a process of making a liner for personal care products involving hydroentangling a hydrophilic first nonwoven layer with a hydrophobic second nonwoven layer and aperturing the layers. The layers may be apertured simultaneously to produce aligned apertures.

### BRIEF DESCRIPTION OF THE FIGS.

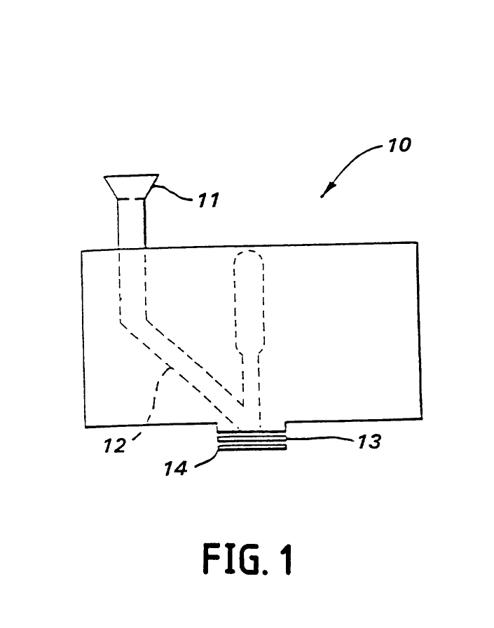

FIG. 1 is a diagram of a rate block used in testing the materials of this invention.

### DEFINITIONS

“Disposable” includes being disposed of after a single use and not intended to be washed and reused. “Hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials can be provided by a Cahn SFA-222 Surface Force Analyzer System, or a substantially equivalent system. When measured with this system, fibers having contact angles less than 90° are designated “wettable” or hydrophilic, while fibers having contact angles equal to or greater than to 90° are designated “nonwettable” or hydrophobic.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface. “Spunbonded fibers” refers to small diameter fibers that are formed by extruding molten thermoplastic material as filaments from a plurality of fine capillaries of a spinneret. Such a process is disclosed in, for example, U.S. Pat. No. 4,340,563 to Appel et al. and U.S. Pat. No. 3,802,817 to Matsuki et al. The fibers may also have shapes such as those described, for example, in U.S. Pat. No. 5,277,976 to Hogle et al. which describes fibers with unconventional shapes.

As used herein “hydroentangling” means a process wherein a nonwoven web, or layers of a non-woven web, are subjected to streams of a non-compressible fluid, e.g., water, at a high enough energy level and for a sufficient time to entangle the fibers thereof. The fluid may advantageously be used at a pressure of between about 200 and 5000 psig (14-351 kg/cm² gauge) from a distance of a few inches (centimeters) above the web while the web is supported by a mesh structure. This process is described in detail in U.S. Pat. No. 3,486,168 to Evans et al. incorporated herein by reference. Nonwoven webs subjected to hydroentangling are referred to as, for example, “spunlace” fabrics. “Bonded carded web” refers to webs that are made from staple fibers which are sent through a combing or carding unit, which separates or breaks apart and aligns the staple fibers in the machine direction to form a generally machine direction-oriented fibrous nonwoven web. This material may be bonded together by methods that include point bonding, through air bonding, ultrasonic bonding, adhesive bonding, etc.

“Airlaying” is a well-known process by which a fibrous nonwoven layer can be formed. In the airlaying process, bundles of small fibers having typical lengths ranging from about 3 to about 52 millimeters (mm) are separated and entrained in an air supply and then deposited onto a forming screen, usually with the assistance of a vacuum supply. The randomly deposited fibers then are bonded to one another using, for example, hot air or a spray adhesive. Airlaying is taught in, for example, U.S. Pat. No. 4,640,810 to Laursen et al. “Personal care product” means products for the absorption of body exudates, such as diapers, training pants, disposable swim wear, absorbent underpants, adult incontinence products, bandages, veterinary and mortuary products, and feminine hygiene products like sanitary napkins and pantiliners.

“Target area” refers to the area or position on a personal care product where an insult is normally delivered by a wearer.

### Test Methods and Materials

Basis Weight: A circular sample of 3 inches (7.6 cm) diameter is cut and weighed using a balance. Weight is recorded in grams. The weight is divided by the sample area. Five samples are measured and averaged.

Material caliper (thickness): The caliper of a material is a measure of thickness and is measured at 0.05 psi (3.5 g/cm²) with a STARRET®) bulk tester, in units of millimeters. Samples are cut into 4 inch by 4 inch (10.2 cm by 10.2 cm) squares and five samples are tested and the results averaged.

Density: The density of the materials is calculated by dividing the weight per unit area of a sample in grams per square meter (gsm) by the material caliper in millimeters (mm). The caliper should be measured at 0.05 psi (3.5 g/cm²) as mentioned above. The result is multiplied by 0.001 to convert the value to grams per cubic centimeter (g/cc). A total of five samples would be evaluated and averaged for the density values.

Absorption Time and Rewet: This test is used to examine the fluid handling properties of a fabric. The following procedure and equipment are used:

1. Stack nonwoven intake test sample on top of fluff pad. Record dry weights, dimensions and thickness of each layer.

2. Center rate block on top of sample. A funnel is placed in a small upper hole in the rate block.

3. Attach pipette tip is attached to Pipetman. Set Pipetman bottle to deliver 6.0 mL of fluid into the funnel on the rate block.

4. Insult 6.0 mL of Z-date simulant to absorbent material using the Pipetman bottle. Use the stopwatch to measure the length of time from delivery of fluid to materials until all fluid is fully absorbed. Record this time as the absorption time.

5. Wait 60 seconds.

6. Remove rate block from sample and fluff pad.

7. Place absorbent material sample and fluff pad on the hot water bottle of the pressure stand. Place two pieces of pre-weighed blotter paper on top of the sample. The test button on the pressure gauge is then depressed, starting a program that applies 1.0 psi of pressure to the system for 3 minutes. At the end of 3 minutes, the pressure stand lowers, releasing the pressure from the absorbent materials.

8. Reweigh the wet blotter papers. Record weights. The moisture pick-up in the blotter reflects the fluid the paper absorbs from the system, in grams.

9. Weigh and check thicknesses on the embossed fluff and nonwoven test sample. Record results.

Equipment Used

Gilson Pipetman P5000, using RC-5000 pipette tips and pipetman filters

Omega Engineering pressure gauge with timer, Model HHP-701-20

Blotter rewet pressure stand with water bottle

Stopwatches

Intake fabric sample materials, approximately 5″×5″

Desorption Material—600 gsm sine-wave embossed fluff

Z-Date fluid, available from the BASF Corp., 2901 North Conkey St., Appleton, Wis.

Plexiglass rateblock (see FIG. 1): The rate block 10 is 3 inches (76.2 mm) wide and 2.87 inches (72.9 mm) deep (into the page) and has an overall height of 1.125 inches (28.6 mm) which includes a center area 19 on the bottom of the rate block 10 that projects farther from the main body of the rate block 10 and has a height of 0.125 inches (3.2 mm) and a width of 0.886 inches (22.5 mm). The rate block 10 has a capillary 12 with an inside diameter of 0.186 inches (4.7 mm) that extends diagonally downward from one side 15 to the center line 16 at an angle of 21.8 degrees from the horizontal. The capillary 12 may be made by drilling the appropriately sized hole from the side 15 of the rate block 10 at the proper angle beginning at a point 0.726 inches (18.4 mm) above the bottom of the rate block 10; provided, however, that the starting point of the drill hole in the side 15 must be subsequently plugged so that test fluid will not escape there. The top hole 17 has a diameter of 0.312 inches (7.9 mm), and a depth of 0.625 inches (15.9 mm) so that it intersects the capillary 12. The top hole 17 is perpendicular to the top of the rate block 10 and is centered 0.28 inches (7.1 mm) from the side 15. The top hole 17 is the aperture into which the funnel 11 is placed. The center hole 18 is for the purpose of viewing the progression of the test fluid and is actually of an oval shape into the plane of FIG. 1. The center hole 18 is centered width-wise on the rate block 10 and has a bottom hole width of 0.315 inches (8 mm) and length of 1.50 inches (38.1 mm) from center to center of 0.315 inch (8 mm) diameter semi-circles making up the ends of the oval. The oval enlarges in size above 0.44 inches (11.2 mm) from the bottom of the rate block 10, for ease of viewing, to a width of 0.395 inches (10 mm) and a length of 1.930 inches (49 mm). The top hole 17 and center hole 18 may also be made by drilling.

### DETAILED DESCRIPTION OF THE INVENTION

Modern personal care products usually have an outer cover, an inner core portion and a liner that goes against the wearer's skin.

The outer cover or “baffle” is designed to be impermeable to liquid in order to keep the clothing or bedding of the wearer from becoming soiled. The impermeable baffle is preferably made from a thin film and is generally made from plastic though other materials may be used. Nonwoven webs, films or film coated nonwovens may be used as the baffle as well. Suitable film compositions for the baffle include polyethylene film which may have an initial thickness of from about 0.5 mil (0.012 millimeter) to about 5.0 mil (0.12 millimeter). The baffle may optionally be composed of a vapor or gas permeable, microporous “breathable” material, that is permeable to vapors or gas yet substantially impermeable to liquid. Breathability can be imparted in polymer films by, for example, using fillers in the film polymer formulation, extruding the filler/polymer formulation into a film and then stretching the film sufficiently to create voids around the filler particles, thereby making the film breathable. Generally, the more filler used and the higher the degree of stretching, the greater the degree of breathability. Other suitable thermoplastic materials like other olefins, nylons, polyesters or copolymers of, for example, polyethylene and polypropylene may also be used.

The core portion of a personal care product is designed to absorb liquids and secondarily to contain solids. The core, known also as an absorbent core, a retention layer, and the like, may be made with pulp and/or superabsorbent materials. These materials absorb liquids quite quickly and efficiently in order to minimize leakage. Core materials may be made according to a number of processes including the coform process, airlaying, and bonding and carding and should be between 50 and 350 gsm.

Various other layers may be included in some personal care products. These include surge layers, usually placed between the liner and core and designed, as the name suggests, to contain large surges of liquid so that the core may absorb it over time. Distribution layers also are included in many personal care products. Distribution layers are usually located next to the core and accept liquid from the surge or liner layer and distribute it to other areas of the core. In this manner, rather than absorbing liquid exclusively in the vicinity of the target area, more of the absorbent core is used.

The liner is designed to be highly permeable to liquid and to be non-irritating to the skin. The liner may optionally have more than one layer or may have one layer in a central area with multiple layers in the side areas. The opposite configuration is also possible with two or more layers in the central area and only one on the sides.

More sophisticated types of liners may incorporate treatments of lotions or medicaments to improve the environment near the skin or to actually improve skin health. Such treatments include aloe, vitamin E, mineral oil, baking soda and other preparations as may be known or developed by those skilled in the art. These treatments are applied to the surface of the liner which will be in contact with the skin of the wearer.

The inventors have found that it is advantageous to have a hydrophilic layer as the outermost bodyside part of the liner, in contact with the wearer. This results in a very rapid absorption of fluids. A hydrophilic liner over an absorbent core, however, will in many cases allow liquid to move upwardly from the core toward the wearer again and “rewet” the skin of the wearer. It will also allow liquid to spread from the target area to the sides of the pad so that the stained area is much larger than that, for example, of a film covered pad. These are regarded as significant negative factors in the design of disposable personal care products since they can result in staining of clothing and bedding, and discomfort to the wearer.

If a hydrophobic layer is placed below the hydrophilic liner, the ability of liquid to move is upwardly from the wetted core is significantly reduced. This results in much better “rewet” values, smaller stain sizes, reduced stain color intensity, and helps keep the wearer drier.

Unfortunately, a hydrophobic layer immediately below the hydrophilic layer also impedes the movement of liquid from the wearer to the absorbent core, causing pooling of liquid on the liner. This can ultimately result in runoff and staining of the clothing and bedding, the very problem that the hydrophobic layer was attempting to solve.

The inventors have solved the problem posed by the hydrophobic layer in two ways;

by aperturing the layers and by joining them using a laminating process involving no chemical or thermal bonding processes.

Aperturing of the hydrophilic layer and hydrophobic layer provides a rapid, open pathway to the absorbent core for liquid from the surface of the liner. This solves the problem posed by the hydrophobic layer's barrier to liquid passage. Once liquid passes through the apertures, it tends to spread out below the hydrophobic layer and go into the absorbent core. Since the apertures are but a small percentage of the surface area of the hydrophilic/hydrophobic liner, the amount of liquid going back upward through them is significantly smaller than the amount of liquid that can pass upwardly through the hydrophilic liner alone.

Aperturing of the laminate may occur after, during or before hydroentangling, which is discussed below, though doing so afterwards is preferred. Aperturing may be carried out by any means known in the art, including using mechanical pin aperturing, by die cutting or by forming the materials in such a way that they are produced with holes in place. The apertures may also be made through the use of high pressure water jets, which may occur while the fabrics are being hydroentangled. The surface area of the liner may be apertured to produce from between 10 and 50 percent open area, more particularly between 20 and 40 percent, and still more particularly about 25 percent.

The use of the hydroentangling process to join the layers, instead of chemical or thermal bonding means, produces a laminate without melted fiber cross over points. This avoids the production of relatively large masses of thermoplastic that can impede fluid movement. High pressure water entangling can also be used to remove a non-durable hydrophilic surface treatment from the hydrophobic layer during processing.

The fibers from which the hydrophilic layer may be made include naturally hydrophilic fibers such as cotton and Rayon, or synthetic fibers that are naturally hydrophobic but which have been treated to be hydrophilic. If the fibers are synthetic fibers treated to be hydrophilic, the treatment must be sufficiently durable to withstand the rigors of hydroentangling. It is not required that all of the fibers of the layer be hydrophilic, just that the layer be predominately hydrophilic. The layer may be made from a blend of fibers.

The fibers from which the hydrophobic layer may be made include naturally hydrophobic fibers like synthetic polymer fibers. It is not required that all of the fibers of the layer be hydrophobic, just that the layer be predominately hydrophobic. The layer may be made from a blend of fibers. As mentioned above, hydroentangling can also be used to remove a previously applied non-durable hydrophilic surface treatment from the hydrophobic layer during processing, thus rendering it hydrophobic again.

The fibrous layers of this invention may be made from any nonwoven process know in the art, including airlaying, spunbonding, meltblowing and carding of staple fibers. The layers may have basis weights from 0.25 to 3 osy (8.5 to 102 gsm) each.

Synthetic fibers include those made from polyolefins, polyamides, polyesters, acrylics, LYOCELL®) regenerated cellulose, Lenzing's viscose rayon, and any other suitable hydrophobic synthetic fibers known to those skilled in the art. Many polyolefins are available for fiber production, for example polyethylenes such as Dow Chemical's ASPUN® 6811A liner low density polyethylene, 2553 LLDPE and 25355 and 12350 high density polyethylene are such suitable polymers. The polyethylenes have melt flow rates, respectively, of about 26, 40, 25 and 12. Fiber forming polypropylenes include Kolon Glotec's T-1001, Exxon Chemical Company's ESCORENE® PD 3445 and Montell Chemical Co.'s PF304. Other polyolefins are also available. Fibers having a lower melting polymer component, like conjugate and biconstituent fibers are suitable for use as well. Such fibers include conjugate fibers of polyolefins, polyamides and polyesters like the sheath core conjugate fibers available from KoSa Inc. (Charlotte, N.C.) under the designation T-255 and T-256.

Natural fibers include wool, cotton, flax, hemp and wood pulp. Wood pulps include standard softwood fluffing grade such as CR-1654 (US Alliance Pulp Mills, Coosa, Ala.). Pulp may be modified in order to enhance the inherent characteristics of the fibers and their processability.

The bodyside layer of this invention is preferably made from a blend of hydrophilic fibers with a minor amount of hydrophobic fibers. The hydrophilic fibers should be present in an amount from about 50 to 100 percent, more particularly from 70 to 100 weight percent and still more particularly 80-100 weight percent.

The layer away from the body should have predominately hydrophobic fibers. The low cost of polypropylene fibers makes it an excellent choice for such a product and polypropylene fibers in an amount of as much as 100 weight percent may be used. Blends of polypropylene with other fibers like PET also function well.

The following helps illustrate the invention.

### EXAMPLE 1

A two layer laminate was made having a top or bodyside facing layer and a bottom or absorbent core facing layer. The top layer was a 0.40 osy (13.5 gsm) carded web and had 90 weight percent Rayon, naturally hydrophilic fiber and 10 weight percent polyethylene terephthalate (PET) fibers. The bottom layer was a 0.47 osy (16.5 gsm) carded web having 73 weight percent PET and 27 weight percent polypropylene (PP) fibers. The layers were hydroentangled at a water pressure of 435-725 psi (30-51 kgf/cm²) and apertured afterwards at a density of approximately 50 apertures per cm² by the hydroentanglement process at 580 psi (41 kgf/cm²). The apertures were approximately 0.06 mm in diameter or about 0.3 mm² in area. The apertures were roughly diamond shaped because the mesh upon which the laminate was supported was diamond shaped. Support media with other shapes would result in other shapes and sizes for the apertures.

Control

A single layer liner having hydrophilic properties. The liner was made from 80 weight percent Rayon and 20 weight percent PET fibers. This liner had a basis weight of 0.89 osy (30 gsm) and was apertured in the same manner and pattern as Example 1.

Example 1 and Control were tested according to the intake rate and rewet tests above and the results given in the Table. It's clear that the liner of this invention had a faster intake rate than that of the control liner. The rewet rate is also better. It was further noted that, in a comparison test using equal amounts of swine blood, the stain size was different for the Control and Example 1. The stain length was about the same, but the shape and width was different, with the Control having a wider, more elliptical shape and Example 1 having a narrower, more rectangular shape.

TABLE

| | Intake Rate(sec) | Rewet (g) |
|---|---|---|
| Control | 14.4 | 1.5 |
| Example 1 | 13.7 | 1.3 |

As will be appreciated by those skilled in the art, changes and variations to the invention are considered to be within the ability of those skilled in the art. Such changes and variations are intended by the inventors to be within the scope of the invention.

What is claimed is:

1) A liner for personal care products comprising a hydrophilic first apertured nonwoven layer laminated with a hydrophobic second apertured nonwoven layer.

2) The liner of claim 1 wherein said first layer and said second layer apertures are aligned.

3) The liner of claim 1 wherein said first layer comprises durably hydrophilic fibers.

4) The liner of claim 1 wherein said second layer comprises non-durably hydrophilic fibers.

5) The liner of claim 1 wherein said layers are laminated according to a spunlace process.

6) The liner of claim 1 further comprising a treatment applied to said hydrophilic layer, said treatment selected from the group consisting of aloe, vitamin E, mineral oil, baking soda and combinations thereof.

7) A liner for personal care products comprising a first nonwoven layer comprising staple, naturally hydrophilic fibers hydroentangled to form a laminate with a second nonwoven layer comprising hydrophobic fibers, wherein said laminate is apertured with an area of 10 to 50 percent.

8) The liner of claim 7 wherein said first layer comprises hydrophilic fibers selected from the group consisting of rayon, pulp, cotton, naturally hydrophilic fibers, and mixtures thereof.

9) The liner of claim 7 wherein said second layer comprises hydrophobic fibers made from polymers selected from the group consisting of polyolefins, polyesters, acrylics and mixtures thereof.

10) The liner of claim 7 wherein said laminate is apertured with an area of from 10 to 50 percent.

11) The liner of claim 7 wherein said apertures in said layers are aligned.

12) A personal care product comprising the liner of claim 11.

13) A pantiliner comprising a liquid permeable liner, a liquid impervious baffle, and an absorbent core positioned therebetween, wherein said liner comprises a hydrophilic first apertured nonwoven layer laminated according to a spunlace process with a hydrophobic second apertured nonwoven layer.

14) The pantiliner of claim 13 wherein said first layer and said second layer apertures are aligned.

15) A process of making a liner for personal care products comprising hydroentangling a hydrophilic first nonwoven layer with a hydrophobic second nonwoven layer and aperturing said layers.

16) The process of claim 15 wherein said layers are apertured simultaneously to produce aligned apertures.
